(19) United States
(12) Patent Application Publication
(10) Pub. No.: US 2022/0202939 A1
(43) Pub. Date: Jun. 30, 2022

(54) PREPARATION FOR IMPROVING ACTIVITY AND/OR THERMAL STABILITY OF SUPEROXIDE DISMUTASE, AND APPLICATION THEREOF

(71) Applicant: CHINA AGRICULTURAL UNIVERSITY, Beijing (CN)

(72) Inventors: Xiaojun LIAO, Beijing (CN); Yang ZHAO, Beijing (CN); Yongtao WANG, Beijing (CN); Liang ZHAO, Beijing (CN); Lei RAO, Beijing (CN)

(21) Appl. No.: 17/699,204

(22) Filed: Mar. 21, 2022

Related U.S. Application Data
(63) Continuation of application No. PCT/CN2021/119098, filed on Sep. 17, 2021.

(30) Foreign Application Priority Data
Sep. 29, 2020 (CN) 202011047414.1

(51) Int. Cl. A61K 47/22; A61K 47/12; A23L 33/15

(52) U.S. Cl. CPC A61K 47/22; A23L 33/15; A61K 47/12

(57) ABSTRACT

The present disclosure provides a preparation for improving activity and/or thermal stability of superoxide dismutase, and an application thereof. The preparation includes at least one of vitamin C, vitamin p, catechin, quercetin, myricetin, kaempherol, ellagic acid, or ferulic acid.

### CROSS-REFERENCE TO RELATED APPLICATIONS

This application is a continuation of International Patent Application No. PCT/CN2021/119098, filed on Sep. 17, 2021, which claims priority to Chinese Patent Application No. 202011047414.1, filed on Sep. 29, 2020, the entire disclosures of which are hereby incorporated by their references.

### TECHNICAL FIELD

The present disclosure relates to the field of biology. More particularly, the present disclosure relates to a preparation for improving activity and/or thermal stability of superoxide dismutase, and applications thereof.

### BACKGROUND

Superoxide dismutase (SOD) is a kind of redox metalloenzymes, which can remove superoxide anion radicals in vivo and effectively prevent damages of superoxide anion radicals to organisms. As the number one killer of oxygen radicals in organisms, the SOD is the foundation of life and health. SOD enzymes are widely distributed in various organisms, such as animals, plants, microorganisms, etc. However, the SOD has disadvantages of low activity and poor thermal stability, which hinder popularization and applications of the SOD.

### SUMMARY

The present disclosure aims to solve at least one of the technical problems in the related art at least to a certain extent. To this end, the present disclosure provides a preparation for improving activity and/or thermal stability of superoxide dismutase, use of the preparation in improving activity of superoxide dismutase, use of the preparation in improving thermal stability of superoxide dismutase, a composition, a method for improving activity of superoxide dismutase, a method for improving thermal stability of superoxide dismutase, and a food, medicine, health care product, or kit. The vitamin C, vitamin p, catechin, quercetin, myricetin, kaempherol, ellagic acid, and ferulic acid contained in the preparation can effectively improve the activity and thermal stability of the superoxide dismutase, and endow the superoxide dismutase with a wide application prospect.

In an aspect of the present disclosure, the present disclosure provides a preparation for improving activity and/or thermal stability of superoxide dismutase. According to embodiments of the present disclosure, the preparation includes at least one of vitamin C, vitamin p, catechin, quercetin, myricetin, kaempherol, ellagic acid, or ferulic acid. Inventors found that the vitamin C, the vitamin p, the catechin, the quercetin, the myricetin, the kaempherol, the ellagic acid, and the ferulic acid can effectively improve the activity and thermal stability of the superoxide dismutase, and endow the superoxide dismutase with a wide application prospect.

In another aspect of the present disclosure, the present disclosure provides use of the above-mentioned preparation in improving activity of superoxide dismutase. The inventors found that the vitamin C, the vitamin p, the catechin, the quercetin, the myricetin, the kaempherol, the ellagic acid, and the ferulic acid can effectively improve the activity of the superoxide dismutase, and endow the superoxide dismutase with a wide application prospect.

In yet another aspect of the present disclosure, the present disclosure provides use of the above-mentioned preparation in improving thermal stability of superoxide dismutase. The inventors found that the vitamin C, the vitamin p, the catechin, the quercetin, the myricetin, the kaempherol, the ellagic acid, and the ferulic acid can effectively improve the thermal stability of the superoxide dismutase, and endow the superoxide dismutase with a wide application prospect.

In still yet another aspect of the present disclosure, the present disclosure provides a composition. According to embodiments of the present disclosure, the composition includes superoxide dismutase, and the above-mentioned preparation. By adding the above-mentioned preparation to the composition, the activity and thermal stability of the superoxide dismutase can be improved, and an application effect of the superoxide dismutase can be enhanced.

According to embodiments of the present disclosure, a concentration ratio of each of the vitamin C, the vitamin p, the catechin, the quercetin, the myricetin, the kaempherol, the ellagic acid, and the ferulic acid to the superoxide dismutase, i.e., a molar ratio of each of the vitamin C, the vitamin p, the catechin, the quercetin, the myricetin, the kaempherol, the ellagic acid, and the ferulic acid to the superoxide dismutase, is independently 1:(1 to 10). The inventors found that a composition prepared with the superoxide dismutase and each of the vitamin C, the vitamin p, the catechin, the quercetin, the myricetin, the kaempherol, the ellagic acid, and the ferulic acid in the above-mentioned ratio can better improve the activity and thermal stability of the superoxide dismutase.

In still yet another aspect of the present disclosure, the present disclosure provides a method for improving activity of superoxide dismutase. According to embodiments of the present disclosure, the method includes contacting the superoxide dismutase with the above-mentioned preparation. The inventors found that the vitamin C, the vitamin p, the catechin, the quercetin, the myricetin, the kaempherol, the ellagic acid, and the ferulic acid can effectively improve the activity of the superoxide dismutase.

According to embodiments of the present disclosure, a concentration ratio of each of the vitamin C, the vitamin p, the catechin, the quercetin, the myricetin, the kaempherol, the ellagic acid, and the ferulic acid to the superoxide dismutase, i.e., a molar ratio of each of the vitamin C, the vitamin p, the catechin, the quercetin, the myricetin, the kaempherol, the ellagic acid, and the ferulic acid to the superoxide dismutase, is independently 1:(1 to 10). The inventors found that contacting each of the vitamin C, the vitamin p, the catechin, the quercetin, the myricetin, the kaempherol, the ellagic acid, and the ferulic acid with the superoxide dismutase in the above-mentioned ratio can better improve the activity of the superoxide dismutase.

In still yet another aspect of the present disclosure, the present disclosure provides a method for improving thermal stability of superoxide dismutase. According to embodiments of the present disclosure, the method includes contacting the superoxide dismutase with the above-mentioned preparation. The inventors found that the vitamin C, the vitamin p, the catechin, the quercetin, the myricetin, the kaempherol, the ellagic acid, and the ferulic acid can effectively improve the thermal stability of the superoxide dismutase.

According to an embodiment of the present disclosure, a concentration ratio of each of the vitamin C, the vitamin p, the catechin, the quercetin, the myricetin, the kaempherol, the ellagic acid, and the ferulic acid to the superoxide dismutase, i.e., a molar ratio of each of the vitamin C, the vitamin p, the catechin, the quercetin, the myricetin, the kaempherol, the ellagic acid, and the ferulic acid to the superoxide dismutase, is independently 1:(1 to 10). The inventors found that contacting each of the vitamin C, the vitamin p, the catechin, the quercetin, the myricetin, the kaempherol, the ellagic acid, and the ferulic acid with the superoxide dismutase in the above-mentioned ratio can better improve the thermal stability of the oxide dismutase.

In still yet another aspect of the present disclosure, the present disclosure provides a food, medicine, health care product, or kit. According to embodiments of the present disclosure, the food, medicine, health care product, or kit includes the above-mentioned composition. The food, medicine, health care product, or kit according to the embodiments of the present disclosure contains the superoxide dismutase with higher activity and higher thermal stability, such that the food, medicine, health care product, or kit according to the embodiments of the present disclosure has a better application effect.

It should be noted that the present disclosure does not strictly limit sources of the vitamin C, the vitamin p, the catechin, the quercetin, the myricetin, the kaempherol, the ellagic acid, and the ferulic acid, and the sources can be food (such as prickly pear, kiwi fruit, apple), microorganisms, or chemical synthesis. For the food, medicine, or health care product, food sources are preferable to improve the safety of use.

Additional aspects and advantages of the present disclosure will be given at least in part in the following description, or become apparent at least in part from the following description, or can be learned from practicing of the present disclosure.

### BRIEF DESCRIPTION OF DRAWINGS

The above and/or additional aspects and advantages of the present disclosure will become more apparent and more understandable from the following description of embodiments taken in conjunction with the accompanying drawings, in which:

FIG. 1 is a schematic diagram showing an analysis of influences of different small-molecule substances on thermal stability of SOD at 80° C. according to an embodiment of the present disclosure.

### DESCRIPTION OF EMBODIMENTS

Solutions of the present disclosure will be explained below in conjunction with the embodiments. It can be appreciated by those skilled in the art that the following embodiments are only used to illustrate the present disclosure, and should not be construed as a limitation on the scope of the present disclosure. Where a specific technique or condition is not indicated in the embodiments, an implementation can be performed in accordance with the technique or condition described in a literature in the field or a product specification. A reagent or instrument used herein without specifying a manufacturer is a conventional product that can be obtained from the market.

### Embodiment 1

In this embodiment, influences of different small-molecule substances on superoxide dismutase were studied.

1. Reagents

Vc solution: 1.8 mg of vitamin C was weighed and dissolved in 10 mL of water to obtain a stock solution of C_{vc}=1×10⁻³ mol/L.

Vp solution: 6.1 mg of vitamin P was weighed and dissolved in 10 mL of ethanol to obtain a stock solution of Cᵥₚ=1×10⁻³ mol/L.

Catechin solution: 2.9 mg of catechin was weighed and dissolved in 10 mL of ethanol to obtain a stock solution of C_{catechin}=1×10⁻³ mol/L.

Quercetin solution: 3.0 mg of quercetin was weighed and dissolved in 10 mL of ethanol to obtain a stock solution of C_{quercetin}=1×10⁻³ mol/L.

Myricetin solution: 3.2 mg of myricetin was weighed and dissolved in 10 mL of ethanol to obtain a stock solution of C_{myricetin}=1×10⁻³ mol/L.

Kaempherol solution: 2.9 mg of kaempherol was weighed and dissolved in 10 mL of ethanol to obtain a stock solution of Cₖₐₑₘₚₕₑᵣₒₗ=1×10⁻³ mol/L.

Ellagic acid solution: 3.0 mg of ellagic acid was weighed and dissolved in 10 mL of ethanol to obtain a stock solution of C_{ellagic acid}=1×10⁻³ mol/L.

Ferulic acid solution: 1.9 mg of ferulic acid was weighed and dissolved in 10 mL of ethanol to obtain a stock solution of C_{ferulic acid}=1×10⁻³ mol/L.

VB1 solution: 3.4 mg of vitamin B1 was weighed and dissolved in 10 mL of water to obtain a stock solution of C_{vb1}=1×10⁻³ mol/L.

SOD solution: Mr(SOD)=37.8 kDa, 37.8 mg of SOD was weighed and dissolved in 10 mL of phosphate buffer to obtain a stock solution of C_{SOD}=1×10⁻⁴ mol/L.

2. Steps

Experimental group 1: 100 μL of the SOD solution was taken out and added to each of five 10 mL centrifuge tubes, and then 100 μL of the solution of each of the vitamin C, the vitamin p, the catechin, the quercetin, the myricetin, the kaempherol, the ellagic acid, and the ferulic acid was added to each of the five centrifuge tubes. Another 100 μL of the SOD solution was taken out as blank control. The centrifuge tubes and the blank control were placed in a water bath at 80° C., incubated for 4 hours, and detected for enzyme activity every 20 minutes.

Experimental group 2: 100 μL of the SOD solution was taken out and added to each of five 10 mL centrifuge tubes, and then 70 μL of the solution of each of the vitamin C, the vitamin p, the catechin, the quercetin, the myricetin, the kaempherol, the ellagic acid, and the ferulic acid was added to each of the five centrifuge tubes. Another 100 μL of the SOD solution was taken out as blank control. The centrifuge tubes and the blank control were placed in a water bath at 80° C., incubated for 4 hours, and detected for enzyme activity every 20 minutes.

Experimental group 3: 100 μL of the SOD solution was taken out and added to each of five 10 mL centrifuge tubes, and then 50 μL of the solution of each of the vitamin C, the vitamin p, the catechin, the quercetin, the myricetin, the kaempherol, the ellagic acid, and the ferulic acid was added to each of the five centrifuge tubes. Another 100 μL of the SOD solution was taken out as blank control. The five 10 mL centrifuge tubes and the blank control were placed in a water bath at 80° C., incubated for 4 hours, and detected for enzyme activity every 20 minutes.

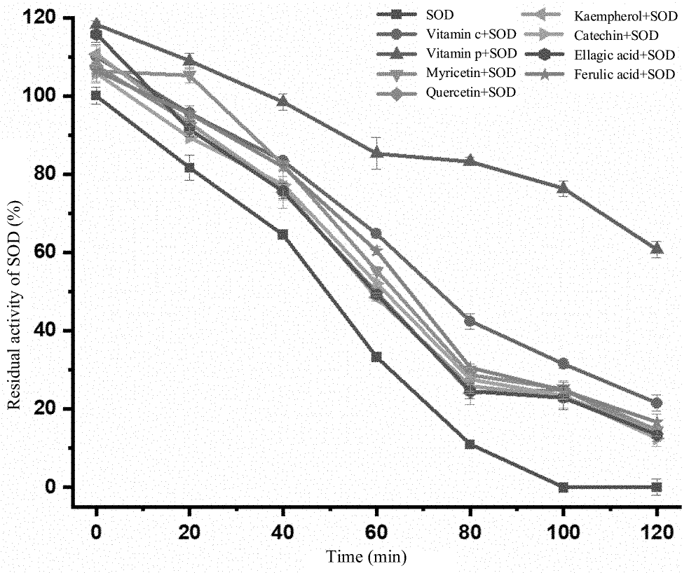

Results of the experimental groups 1 to 3 are similar, and the following analysis is made by only taking experimental data of the experimental group 1 as an example. A result is illustrated in FIG. 1. It can be seen that an addition of each of the vitamin C, the vitamin p, the catechin, the quercetin, the myricetin, the kaempherol, the ellagic acid, and the ferulic acid can improve the activity of the SOD, and significantly improve the thermal stability of the SOD, while Vb1 fails to improve the activity and thermal stability of SOD enzymes (a curve of Vb1, which is not illustrated in the FIGURE, almost coincides with a curve of the SOD in a control group).

Reference throughout this specification to “an embodiment,” “some embodiments,” “an example,” “a specific example,” or “some examples,” means that a particular feature, structure, material, or characteristic described in connection with the embodiment or example is included in at least one embodiment or example of the present disclosure. The appearances of the above phrases in various places throughout this specification are not necessarily referring to the same embodiment or example. Furthermore, the particular features, structures, materials, or characteristics described may be combined in any suitable manner in one or more embodiments or examples. In addition, different embodiments or examples and features of different embodiments or examples described in the specification may be combined by those skilled in the art without mutual contradiction.

Although embodiments of the present disclosure have been shown and described above, it should be understood that above embodiments are merely exemplary, and cannot be construed as limiting the present disclosure. For those skilled in the art, changes, modifications, substitutions, and variations can be made to the embodiments without departing from the scope of the present disclosure.

What is claimed is:

1. A preparation for improving activity and/or thermal stability of superoxide dismutase, comprising at least one of vitamin C, vitamin p, catechin, quercetin, myricetin, kaempherol, ellagic acid, or ferulic acid.

2. Use of the preparation according to claim 1 in improving activity of superoxide dismutase.

3. Use of the preparation according to claim 1 in improving thermal stability of superoxide dismutase.

4. A composition, comprising:
superoxide dismutase; and
the preparation according to claim 1.

5. The composition according to claim 4, wherein a concentration ratio of each of the vitamin C, the vitamin p, the catechin, the quercetin, the myricetin, the kaempherol, the ellagic acid, and the ferulic acid to the superoxide dismutase is independently 1:(1 to 10).

6. A method for improving activity of superoxide dismutase, comprising:
contacting the superoxide dismutase with the preparation according to claim 1.

7. The method according to claim 6, wherein a concentration ratio of each of the vitamin C, the vitamin p, the catechin, the quercetin, the myricetin, the kaempherol, the ellagic acid, and the ferulic acid to the superoxide dismutase is independently 1:(1 to 10).

8. A method for improving thermal stability of superoxide dismutase, comprising:
contacting the superoxide dismutase with the preparation according to claim 1.

9. The method according to claim 8, wherein a concentration ratio of each of the vitamin C, the vitamin p, the catechin, the quercetin, the myricetin, the kaempherol, the ellagic acid, and the ferulic acid to the superoxide dismutase is independently 1:(1 to 10).

10. A food, medicine, health care product, or kit, comprising the composition according to claim 4.

11. A food, medicine, health care product, or kit, comprising the composition according to claim 5.
